(19) United States
(12) Patent Application Publication
(10) Pub. No.: US 2022/0095624 A1
(43) Pub. Date: Mar. 31, 2022

(54) NITRIDE BASED ANTIPATHOGENIC COMPOSITIONS AND DEVICES AND METHODS OF USE THEREOF

(71) Applicant: SINTX Technologies, Inc., Salt Lake City, UT (US)

(72) Inventors: Bryan J. McEntire, Salt Lake City, UT (US); Ryan M. Bock, Salt Lake City, UT (US); Bhajanjit Singh Bal, Salt Lake City, UT (US)

(21) Appl. No.: 17/546,837

(22) Filed: Dec. 9, 2021

Related U.S. Application Data
(63) Continuation-in-part of application No. 17/230,395, filed on Apr. 14, 2021.
Application No. 17/230,395 is a continuation-in-part of application No. 16/550,605, filed on Aug. 26, 2019, now Pat. No. 11,192,787.
(60) Provisional application No. 63/123,037, filed on Dec. 9, 2020.
(60) Provisional application No. 63/045,355, filed on Jun. 29, 2020.
(60) Provisional application No. 62/727,724, filed on Sep. 6, 2018.
(60) Provisional application No. 62/800,034, filed on Feb. 1, 2019.

(51) Int. Cl. A01N 59/00; A01N 59/06; A01N 59/14; A61K 33/06; A61K 33/22; A61K 33/00

(52) U.S. Cl. CPC A01N 59/00; A01N 59/06; A61K 33/00; A61K 33/06; A61K 33/22; A01N 59/14

(57) ABSTRACT

Described herein are antipathogenic compositions comprising a nitride chosen from aluminum nitride, boron nitride, chromium nitride, cerium nitride, hafnium nitride, lanthanum nitride, phosphorous nitride, sulfur nitride, tantalum nitride, titanium nitride, vanadium nitride, yttrium nitride, zirconium nitride, silicon nitride, or combinations thereof, and methods of using said compositions to inactivate viruses, bacteria, and/or fungi.

### CROSS-REFERENCE TO RELATED APPLICATIONS

This application claims priority to U.S. Provisional Application No. 63/123,037, filed Dec. 9, 2020. This application is a continuation-in-part of U.S. application Ser. No. 17/230,395 filed Apr. 14, 2021, which claims priority to U.S. Provisional Application No. 63/045,355 filed on Jun. 29, 2020, and which is a continuation-in-part of U.S. application Ser. No. 16/550,605, now U.S. Pat. No. 11,192,787 filed Aug. 26, 2019, which claims priority to U.S. Provisional Application No. 62/800,034 filed Feb. 1, 2019, and U.S. Provisional Application No. 62/727,724 filed Sep. 6, 2018. The entirety of each of the above applications is incorporated by reference herein.

### FIELD

The present disclosure generally relates to antipathogenic compositions and devices, and in particular to systems and methods for nitride-based antipathogenic compositions and devices.

### BACKGROUND

It is well-known that surface transmission of pathogenic organisms leads to a large number of illnesses, disabilities, and deaths. In recent years, the discovery of new pathogens, such as the SARS-CoV-2 virus, have highlighted this problem. Methods of killing or inactivating pathogens living on surfaces often involve the use of cleaning products, high heat or pressure, or electromagnetic radiation. These methods can be costly and, in some cases, can present other dangers to humans, such as exposure to toxic chemicals or intense ultraviolet radiation. Moreover, some pathogens over time show resistance to existing methods.

There is a need, therefore, for methods and compositions useful for inactivating or killing pathogenic organisms.

### SUMMARY

Provided herein are nitride based compositions or devices for inactivating viruses, bacteria, and/or fungi. The nitride based compositions comprise aluminum nitride, boron nitride, chromium nitride, cerium nitride, hafnium nitride, lanthanum nitride, phosphorus nitride, sulfur nitride, tantalum nitride, titanium nitride, vanadium nitride, yttrium nitride, zirconium nitride, various forms of silicon nitride, or combinations thereof.

Also provided herein are methods for inactivating pathogens by contacting a virus, bacteria, and/or fungus with an antipathogenic nitride based composition or device disclosed herein.

### BRIEF DESCRIPTION OF THE DRAWINGS

FIGS. 1A-1D are graphical representations that show the inactivation of SARS-CoV-2 by nitride powders treated with 15 wt. % Cu, AlN, and Si₃N₄ powders in an aqueous medium at room temperature for 1- and 10-minutes in which the control virus was treated identically without addition of any powders. After centrifugation, supernatants were subjected to TCID₅₀ assay. The Reed-Muench method was used to determine the virus titers. TCID₅₀/50 μL (FIGS. 1A and 1B) and % reduction FIGS. 1C and 1D are shown for virus inactivation times of 1 minutes and 10 minutes, respectively. Statistics are given in the inset according to unpaired two-tailed Student's t-test (n=3).

FIGS. 2A-2D are graphical representations that show viral RNA that has underwent severe degradation after exposure to copper or nitride particles. In FIG. 2A and FIG. 2B, virus suspensions were exposed to Cu, AlN and Si₃N₄ powders for 1 minute, and viral RNA in supernatants and on particles were evaluated using viral N gene “set 1” and “set 2” primers, respectively. Data collected on supernatants and pellet samples are given in comparison with the amount of viral N gene RNA in suspension that was left untreated. In FIG. 2C and FIG. 2D, results of RT-PCR tests after 10-minute exposure of supernatants to Cu, AlN and Si₃N₄ powders for viral N gene “set 1” and “set 2” primers are shown, respectively. Statistics are given in the inset according to unpaired two-tailed Student's t-test (n=3).

FIGS. 3A-3E are images showing Si₃N₄ suppressed virus infection without affecting cell viability in which Cu killed the cells. VeroE6/TMPRSS2 cells were inoculated with (FIG. 3A) unexposed virions, and virions 10-minute UTE-exposed to Si₃N₄ (FIG. 3B), AlN (FIG. 3C), and Cu (FIG. 3D). In FIG. 3E, non-inoculated cells (labeled as “sham-infected” cells) were also prepared and imaged for comparison. After fixation, cells were stained with anti-SARS coronavirus envelope antibody (red), Phalloidin to visualize F-actin (green), and DAPI to stain nuclei (blue). Fluorescence micrographs are shown, which are representative of n=3 samples.

FIG. 4 is a graphical representation showing fluorescently labeled and non-labeled cells were counted on fluorescence micrographs, and % infected cells and % viable cells calculated as follows: % infected cells=(number of the cells stained with anti-SARS coronavirus envelope antibody)/(number of cells stained with DAPI)×100; and, % viable cells=(number of the cells stained with Phalloidin)/(number of cells stained with DAPI)×100. Data is representative of n=3 samples. * and ** are p<0.05 and 0.01, respectively, by unpaired two-tailed Student's t-test (n=3); n.s.=non-significant.

FIGS. 5A-5G are graphical representations of Raman spectra for: (a) uninfected cells (FIG. 5A) (i.e., unexposed to virions), and cells infected with SARS-CoV-2 virions exposed for 10 minutes to (b) Si₃N₄ (FIG. 5B), (c) AlN (FIG. 5C), and (d) Cu (FIG. 5D); in FIG. 5E, Raman spectrum of cells infected by unexposed virions (negative control). In FIG. 5F, a plot of the average intensity of the two tryptophan T1 and T2 bands (at 756 and 875 cm-1, respectively) as a function of fraction of infected cells by virions unexposed and exposed for 10-min to different particles (cf. labels); in the inset, the structure of N′-formylkynurenine, an intermediate in the catabolism of tryptophan upon enzymatic IDO reaction. In FIG. 5G, a graphical representation shows three possible conformations of tyrosine-based peptides that can justify the disappearance of ring vibrations in tyrosine (Ty2 band) upon chelation of Cu(II) ions.

FIG. 6 is a schematic model illustrating a chemical and electrical charge similarity between the protonated amine groups, Si—NH₃⁺, at the surface of Si₃N₄ and the N-terminal of lysine, C—NH₃⁺ in cells (left panel); and, the interaction of SARS-CoV-2 viruses with the charged molecular species at the surface of Si₃N₄ (specifically, at protonated amines charging plus) and the eluted species NH₃/NH₄⁺ (central panel). The eluted N leaves 3+ charged vacancies on the solid surface (violet-colored sites), which stem together with negatively charged silanols. The three-step process leading to RNA backbone cleavage by the eluted nitrogen species (namely, deprotonation of 2′-hydroxyl groups, formation of a transient pentaphosphate, and cleavage of the phosphodiester bond in the RNA backbone by alkaline transesterification through hydrolysis) is shown in the right panel. Note that the similarity between protonated amine and N-terminal of lysine might trigger an extremely effective “competitive binding” mechanism for SARS-CoV-2 virion inactivation, while eluted ammonia fatally degrades the virion RNA in a combined “catch and kill” effect.

FIG.

FIG. 7 is a logarithmic comparison of average bacterial growth on PEEK, boron nitride, aluminum nitride, Shapal (a combination of boron nitride and aluminum nitride), and silicon nitride materials at 24 hours and 48 hours.

FIG. 8 is a chart showing RT-qPCR genomic testing of the Washington State variant of the SARS-CoV-2 virus versus α- and β-silicon nitride powders at 15 wt. %/vol for 30 min exposure.

FIG. 9A is a chart showing plaque assay test results of the Washington State variant of the SARS-CoV-2 virus versus α- and β-silicon nitride powders at 15 wt. %/vol for 30 min exposure.

FIG. 9B is a chart showing plaque assay test results of the South African variant of the SARS-CoV-2 virus versus α- and β-silicon nitride powders at 15 wt. %/vol for 30 min exposure.

FIG. 9C is a chart showing plaque assay test results of the British variant of the SARS-CoV-2 virus versus α- and β-silicon nitride powders at 15 wt. %/vol for 30 min exposure.

Corresponding reference characters indicate elements among the view of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and figures are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, “inactivate” or “inactivation” refers to viral inactivation in which the virus is stopped from contaminating the product or subject either by removing virus completely or rendering them non-infectious.

The terms “object”, “apparatus” or “component” as used herein include materials, compositions, devices, surface coatings, and/or composites. In some examples the apparatus may include various medical devices or equipment, examination tables, clothing, filters, personal protective equipment such as masks and gloves, catheters, endoscopic instruments, commonly-touched surfaces where viral persistence may encourage the spread of disease, and the like. The apparatus may be metallic, polymeric, and/or ceramic (ex. silicon nitride and/or other ceramic materials).

As used herein, “contact” means in physical contact or within close enough proximity to a composition or apparatus to be affected by the composition or apparatus.

As used herein, “personal protective equipment” or “PPE” means any device, article, or apparatus worn or otherwise used by a person to minimize exposure to pathogens or other harmful substances. Non-limiting examples of PPE include body covers, head covers, shoe covers, face masks, eye protectors, face and eye protectors, and gloves.

As used herein, the term “silicon nitride” includes α-Si₃N₄, β-Si₃N₄, SiYAlON, β-SiYAlON, SiYON, SiAlON, or combinations thereof.

As used herein, the term “component” includes a nitride based material, a compound, an implant, a device, or similar, that is useful for antipathogenic purposes.

As used herein, the term “effective concentration” is defined as the concentration of a material required to inactivate at least 90% of a pathogen in at least 30 min. As a non-limiting example, the effective concentration of α-Si₃N₄ may be a concentration which results in at least a 1-log₁₀ reduction in the activity of a virus within 30 min.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

### I. Inactivation of SARS-CoV-2 Using Silicon Nitride and Aluminum Nitride

Provided herein is a method for inactivating the SARS-CoV-2 virus by contacting the virus with an object or composition comprising silicon nitride and/or aluminum nitride. The silicon nitride and/or aluminum nitride successively binds (i.e. captures) and then inactivates the virus (e.g. “catch and kill”).

Silicon nitride possesses a unique surface chemistry which is biocompatible and provides a number of biomedical applications including 1) concurrent osteogenesis, osteoinduction, osteoconduction, and bacteriostasis, such as in spinal and dental implants; 2) killing of both gram-positive and gram-negative bacteria according to different mechanisms; 3) inactivation of human and animal viruses, bacteria, and fungi; and 4) polymer- or metal-matrix composites, natural or manmade fibers, polymers, or metals containing silicon nitride powder retain key silicon nitride bone restorative, bacteriostatic, antiviral, and antifungal properties.

Silicon nitride (Si₃N₄) is a non-oxide ceramic compound that has been used in many industries since the 1950s. A formulation of Si₃N₄ is FDA-cleared for use as an intervertebral spinal spacer in cervical and lumbar spine fusion surgery, with proven long-term safety, efficacy, and biocompatibility. Clinical data for Si₃N₄ implants compare favorably with other spine biomaterials, such as allograft, titanium, and polyetheretherketone. A curious finding is that Si₃N₄ implants have a lower incidence of bacterial infection (i.e., less than 0.006%) when compared to other implant materials (2.7% to 18%). This property reflects the complex surface biochemistry of Si₃N₄ that elutes minute amounts of nitrogen, which is converted to ammonia, ammonium, and other reactive nitrogen species (RNS) that inhibit bacteria. A recent investigation also found that viral exposure to sintered Si₃N₄ powders in aqueous suspension neutralized H1N₁ (Influenza A/Puerto Rico/8/1934), Feline calicivirus, and Enterovirus (EV-A71). Based on these findings, Si₃N₄ may be able to inactivate SARS-CoV-2.

Silicon nitride may be antipathogenic due to release of nitrogen containing species when in contact with an aqueous medium, or biologic fluids and tissues. The surface chemistry of silicon nitride may be shown as follows:

Si₃N₄+6H₂O→3SiO₂+4NH₃

SiO₂+2H₂O→Si(OH)₄

Nitrogen elutes faster (within minutes) than silicon because surface silanols are relatively stable. For viruses, it was surprisingly found that silicon nitride may provide for RNA cleavage via alkaline transesterification which leads to loss in genome integrity and virus inactivation. This may also reduce the activity of hemagglutinin. The elution of ammonia, along with an attendant increase in pH, inactivates viruses, bacteria, and fungi. As shown in the examples, it was surprisingly found that each of silicon nitride and aluminum nitride inactivates SARS-CoV-2.

The use of copper (Cu), a historically-recognized viricidal agent, is limited by its cell toxicity. In contrast to Cu, ceramic devices or apparatuses made of Si₃N₄ are biocompatible and not toxic to the human body. An advantage of Si₃N₄ is the versatility of the material; thus Si₃N₄ may be incorporated into polymers, bioactive glasses, and even other ceramics to create composites and coatings that retain the favorable biocompatible and antiviral properties of Si₃N₄.

The present disclosure compares the effects of exposing SARS-CoV-2 to aqueous suspensions of Si₃N₄ and aluminum nitride (AlN) particles and two controls, (i.e., a suspension of copper (Cu) particles (positive control) and a sham suspension of SARS-CoV-2 virions without any antiviral agent (negative control)). Copper (Cu) was chosen as a positive control because of its well-known ability to inactivate a variety of microbes, including viruses. Aluminum nitride was included in the testing because, like Si₃N₄, it is a nitrogen-based compound whose surface hydrolysis in aqueous solution leads to the elution of nitrogen, with an attendant increase in pH. Since comparable antiviral and antibacterial phenomena are believed to be operative for all nitride-based compounds, AlN was used to provide additional insight into the antipathogenic mechanisms of nitrogen-containing inorganic materials.

The persistence of human coronaviruses on common materials (e.g., metal, plastic, paper, and fabric) and touch surfaces (e.g., knobs, handles, railings, tables, and desktops) can contribute to the nosocomial and social spread of disease. Warnes et al. reported that at room temperature with 30%-40% humidity, the pathogenic human coronavirus 229E (HuCoV-229E) remained infectious in a lung cell model after at least 5 days of persistent viability on a variety of materials, such as Teflon, polyvinyl chloride, ceramic tile, glass, stainless steel, and silicone rubber. These investigators also showed rapid HuCoV-229E inactivation (within a few minutes) for simulated fingertip contamination on Cu surfaces. Cu ion release and the generation of reactive oxygen species (ROS) were involved in viral inactivation; and increased contact time with copper and brass surfaces led to greater non-specific fragmentation of viral RNA, indicating irreversible viral inactivation. More recently, Doremalen et al. showed surface stability of both SARS-CoV-1 and SARS-CoV-2 virus on plastic, cardboard, stainless steel, and even Cu surfaces for 4-72 hours after application. While breathable N₉₅-rated masks can capture particulates before they can be inhaled, SARS-CoV-2 virus particles remain active in mask filters for up to 7 days. Contact killing of viruses, such as observed on Cu surfaces is, therefore, receiving renewed interest as a disease mitigation strategy.

Surprisingly, compounds capable of endogenous nitrogen-release, such as Si₃N₄ and AlN, can inactivate the SARS-CoV-2 virus at least as effectively as Cu. Without being limited to any one theory, multiple antiviral mechanisms may be operative, such as RNA fragmentation, and in the case of Cu and AlN, direct metal ion toxicity; but while Cu and AlN supernatants demonstrated cellular lysis, Si₃N₄ may provoke no metabolic alterations. The Raman spectrum of VeroE6 cells exposed to the Si₃N₄ viral supernatant was like that of the uninfected sham. These findings indicate that while Si₃N₄, Cu, and AlN were all capable of inactivating the SARS-CoV-2 virus, Si₃N₄ was the safest.

The antiviral effect may be related to the electrical attraction (including “competitive binding” to an envelope glycoprotein hemagglutinin in the case of influenza virus) and viral RNA fragmentation by reactive nitrogen species (RNS). These phenomena are due to the slow and controlled elution of nitrogen from Si₃N₄'s surface which forms ammonia (NH₃) and ammonium (NH₄⁺) moieties coupled with the release of free electrons and negatively charged silanols in aqueous solution.

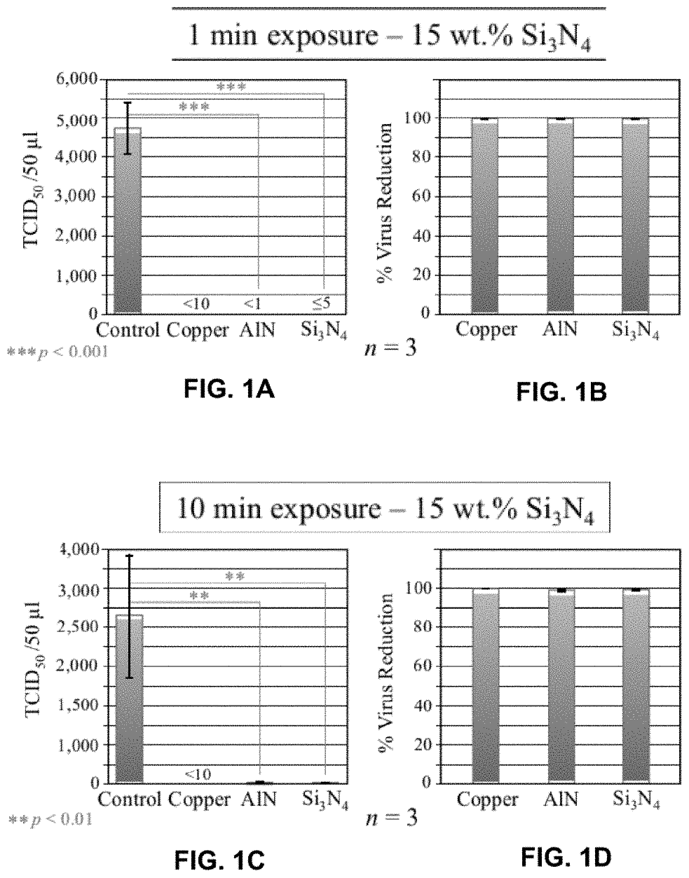

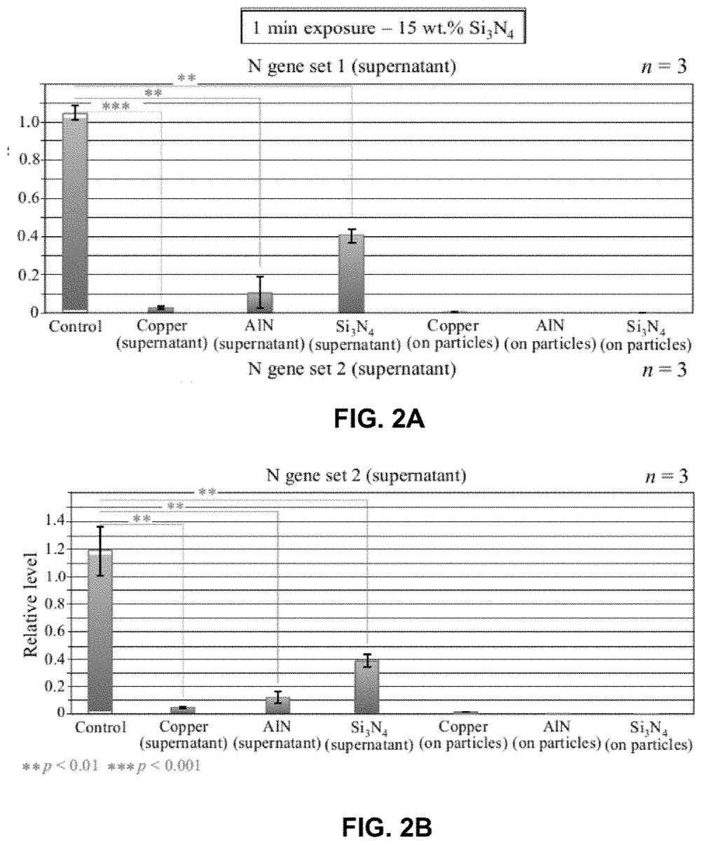

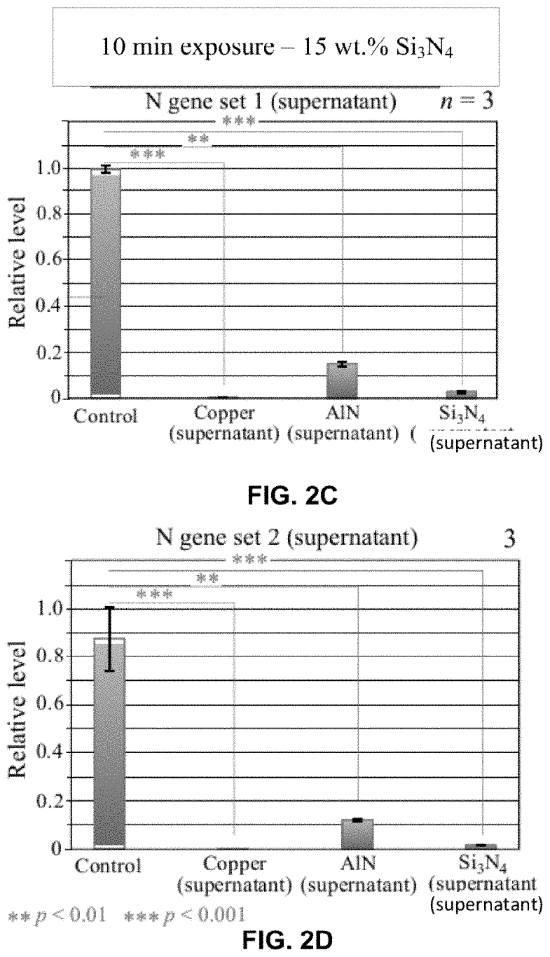

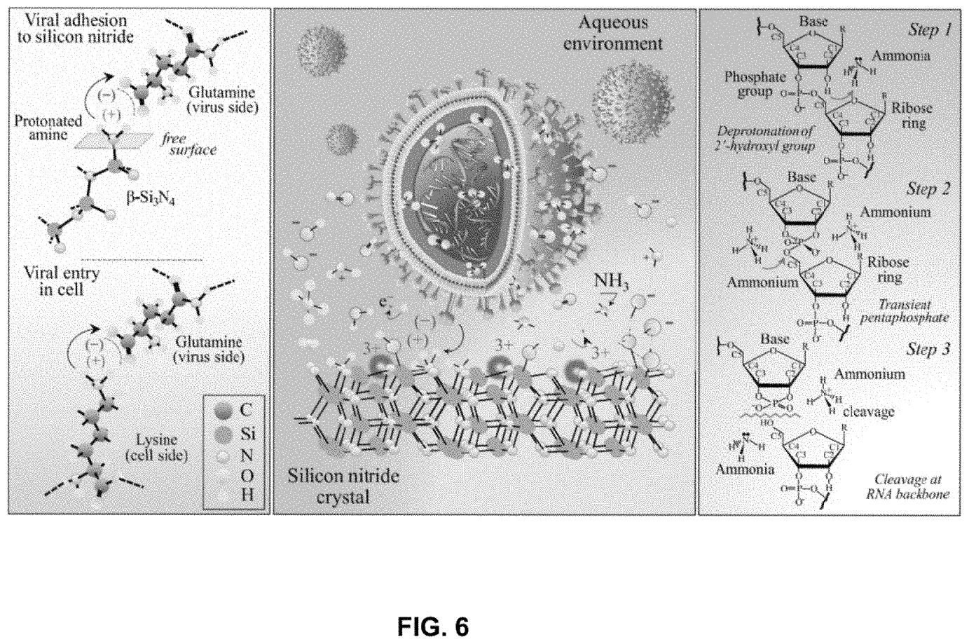

In the context of SARS-CoV-2 viral inactivation, two important aspects of Si₃N₄'s surface chemistry play fundamental roles: (i) the similarity between the protonated amino groups, Si—NH₃⁺ at the surface of Si₃N₄ and the N-terminal of lysine, C—NH₃₊ on the virus; and, (ii) the elution of gaseous ammonia due to Si₃N₄ hydrolysis. A schematic representation of the interaction between SARS-CoV-2 and the Si₃N₄ surface is given in FIG. 6 (central panel). The similarity is depicted in the left panel of this figure. It triggers an extremely effective “competitive binding” approach to SARS-CoV-2 inactivation which stems from several successful other examples such as Hepatitis B and Influenza A. The strong antiviral effect of eluted (gaseous) NH₃ is due to its penetration of the virions and its reaction with the RNA backbone. The RNA undergoes alkaline transesterification through the hydrolysis of its phosphodiester bonds. RNA phosphodiester bond cleavage is schematically depicted in the right panel of FIG. 6. The RT-PCR and fluorescence microscopy results of the present study suggest contributions from both mechanisms to the inactivation of SARS-CoV-2, consistent with earlier work. The TCID₅₀ results shown in FIGS. 1A-1D and the RT-PCR data of FIGS. 2A-2D for viral RNA harvested from either the supernatant or the Si₃N₄ particles provide important information about these mechanisms. Although >99% inactivation was achieved after exposure to Si₃N₄ for 1-min, (FIG. 1B), only partial viral RNA fragmentation was observed for the supernatant (FIG. 2A) while RNA harvested from the Si₃N₄ particles (FIG. 2B) was essentially fully fragmented. Note that the opposite effect was found for Cu. This suggests that the mechanism of inactivation for Si₃N₄, as depicted in the left panel of FIG. 6, had successive events of “competitive binding” and ammonia poisoning—a kind of “catch and kill” scenario. The complete RNA fragmentation at 10-minute exposure to Si₃N₄ suggests that nitrogen elution is a key process that triggers a cascade of reactions which result in virus inactivation (cf. right panel in FIG. 6)

In some embodiments, an object, article, or composition comprising silicon nitride or aluminum nitride may be operable to successively bind a virus (e.g. SARS-Cov-2) and then inactivate the virus.

The antiviral effectiveness of Si₃N₄ may be comparable to Cu. While Cu is an essential trace element for human health and an electron donor/acceptor for several key enzymes by altering redox states between Cu⁺ and Cu²⁺, these properties can also cause cellular damage. Its use as an antiviral agent is limited by allergic dermatitis, hypersensitivity, and multi-organ dysfunction. In contrast, the safety of Si₃N₄ as a permanently-implanted material during spine fusion surgery is well established by experimental and clinical data. Therefore, an object, article, or composition comprising silicon nitride may be as effective at inactivating a virus as Cu without the negative effects of Cu.

Si₃N₄ is well-known for its capabilities as an industrial material. Load-bearing Si₃N₄ prosthetic hip bearings and spinal fusion implants were initially developed because of the superior strength and toughness of Si₃N₄. Later studies showed other properties of Si₃N₄ that are favored in designing orthopaedic implants, such as enhanced osteoconductivity, bacteriostasis, improved radiolucency, lack of implant subsidence, and wear resistance. Therefore, Si₃N₄'s surface chemistry, topography, and hydrophilicity contribute to a dual effect (i.e., upregulation of osteogenic activity to promote spinal fusion while simultaneously preventing bacterial adhesion and biofilm formation). In addition to its proven record as a bioimplant, an advantage of Si₃N₄ is its versatility of manufacture. Sintered powders of Si₃N₄ have been incorporated into other materials, such as polymers, other ceramics, bioglass, and metals, to create composite structures that maintain the index osteogenic and antibacterial properties of monolithic Si₃N₄. Three-dimensional additive deposition of Si₃N₄ may enable the manufacture of protective surfaces in health care that reduce fomite-mediated transmission of microbial disease. Incorporation of Si₃N₄ particles into the fabric of personal protective equipment, such as face masks, protective gowns, and surgical drapes could contribute to health workers as well as patient safety.

Si₃N₄ inactivates the SARS-CoV-2 virus in a matter of minutes following exposure. Without being limited to any one theory, the mechanism of action may be shared with other nitrogen-based compounds that express trace amounts of surface disinfectants, such as aluminum nitride.

In some embodiments, the object used to bind and inactivate the SARS-CoV-2 virus is a device or apparatus that may include a silicon nitride and/or aluminum nitride composition on at least a portion of a surface of the object. The silicon nitride or aluminum nitride coating may be applied to the surface of the object as a powder. In some examples, the silicon nitride or aluminum nitride powder may be filled, embedded, or impregnated in at least a portion of the object. In some embodiments, the powder may have particles in the micron, submicron or nanometer size range. The average particle size may range from about 100 nm to about 5 μm, from about 300 nm to about 1.5 μm, or from about 0.6 μm to about 1.0 μm. In other embodiments, the silicon nitride or aluminum nitride may be incorporated into the device. For example, an object may incorporate a silicon nitride and/or aluminum nitride powder within the body of the object. In one embodiment, the device may be made of silicon nitride. In another embodiment, the object may be made of aluminum nitride. In yet another embodiment, the object can comprise a slurry or suspension of aluminum nitride or silicon nitride particles.

In some embodiments, the object may further comprise other materials including, but not limited to, paper, cardboard, fabric, plastic, ceramic, polymers, stainless steel, metal, or a combination thereof. Some non-limiting examples of the object may include surgical gowns, surgical drapes, shoe covers, cubicle curtains, tubing, clothing, gloves, eye protectors, masks including surgical masks and face shields, PPE, tables such as hospital exam and surgical tables, chairs, bed frames, bed trays, desks, fixtures, cabinets, equipment racks, carts, handles, knobs, railings, toys, water filters, and air filters such as face mask filters, respirator filters, air filtration filters, and air ventilation filters, or air conditioner filters. In some examples, the filters may be within filtration devices of anesthesia machines, ventilators, or CPAP machines such that an antimicrobial surface layer in the filter can trap pulmonary pathogens, as air moves in and out of infected lungs. In various embodiments, the object may be a medical device or apparatus. Non-limiting examples of medical devices or apparatuses include orthopedic implants, spinal implants, pedicle screws, dental implants, in-dwelling catheters, endotracheal tubes, colonoscopy scopes, and other similar devices.

In other embodiments, the object may be a composition incorporating silicon nitride or aluminum nitride powder therein including, but not limited to slurries, suspensions, gels, sprays, paint, or toothpaste. For example, the addition of silicon nitride or aluminum nitride to a slurry, such as paint, that is then applied to a surface may provide an antibacterial, antifungal, and antiviral surface. In other embodiments, silicon nitride or aluminum nitride may be mixed with water along with any appropriate dispersants and slurry stabilization agents, and thereafter applied by spraying the slurry onto various surfaces. An example dispersant is Dolapix A88.

The silicon nitride or aluminum nitride coating may be present on the surface of the object in a concentration of about 1 wt. % to about 100 wt. %. The silicon nitride and/or aluminum nitride may be coated onto or layered into the object. In various embodiments, the coating may include about 1 wt. %, 2 wt. %, 5 wt. %, 7.5 wt. %, 8.3 wt. %, 10 wt. %, 15 wt. %, 16.7 wt. %, 20 wt. %, 25 wt. %, or about 30 wt. % silicon nitride powder or aluminum nitride powder. In some examples, the coating may include about 10 wt. % to about 20 wt. % silicon nitride or aluminum nitride. In at least one example, the coating includes about 15 wt. % silicon nitride or aluminum nitride. In some embodiments, silicon nitride or aluminum nitride may be embedded in (as a filler) or on the surface of the object in a concentration of about 1 wt. % to about 100 wt. %. In various embodiments, the object may include about 1 wt. %, 2 wt. %, 5 wt. %, 7.5 wt. %, 8.3 wt. %, 10 wt. %, 15 wt. %, 16.7 wt. %, 20 wt. %, 25 wt. %, 30 wt. %, 33.3 wt. %, 35 wt. %, 40 wt. %, 50 wt. %, 60 wt. %, 70 wt. %, 80 wt. %, 90 wt. %, to 100 wt. % silicon nitride or aluminum nitride. In some examples, the silicon nitride or aluminum nitride may be on the surface of the object at a concentration of about 10 wt. % to about 20 wt. %. In at least one example, the silicon nitride or aluminum nitride may be on the surface of the object at a concentration of about 15 wt. %. In some aspects, the concentration of silicon nitride or aluminum nitride may depend on the substrate material of the object, such as paper, cardboard, fabric, plastic, ceramic, polymers, stainless steel, and/or metal. In some embodiments, the substrate material of the object may be a polymer and the polymer may have a practical limit (i.e. percolation limit) on the amount of silicon nitride and/or aluminum nitride that may be incorporated into the object.

In some embodiments, the object may be a monolithic component consisting of the silicon nitride or aluminum nitride. Such an object may be fully dense possessing no internal porosity, or it may be porous, having a porosity that ranges from about 1% to about 80%. The monolithic object may be used as a medical device or may be used in an apparatus in which the inactivation of a virus may be desired.

In some embodiments, the object may contact the SARS-CoV-2 virus for a limited period of time. The object may be in contact with the SARS-CoV-2 virus for about 1 min to about 2 hours in order to inactivate the virus. In various examples, the object may contact the SARS-CoV-2 virus for at least 30 seconds, at least 1 minute, at least 5 minutes, at least 30 minutes, at least 1 hour, at least 2 hours, at least 5 hours, or at least 1 day. In at least one example, the object may be permanently implanted in the patient. In at least one example, the object may be worn externally by a user. In another example, the object may be permanently implanted in the patient. In yet another example, the object may be a high contact surface. In further examples, the object may be in continuous or sustained contact with a body fluid of a patient. The body fluid may be blood or gas (e.g., inhalation or exhalation gas).

In some embodiments, the virus is at least 70% inactivated, at least 75% inactivated, at least 80% inactivated, at least 85% inactivated, at least 90% inactivated, at least 95% inactivated, or at least 99% inactivated after contact with the object for at least 1 minute, at least 5 minutes, or at least 30 minutes. In at least one example, the virus is at least 85% inactivated after contact with object for at least 1 minute. In another example, the virus is at least 99% inactivated after contact with the object for at least 30 minutes. In yet another example, the virus is at least 99% inactivated after contact with the object for at least 1 minute.

Also presented herein is an article of personal protective equipment having antiviral and antimicrobial properties. The article may comprise silicon nitride or aluminum nitride incorporated into the article or the silicon nitride or aluminum nitride may be coated onto the surface of the article.

The silicon nitride or aluminum nitride coating may be present on the surface of the article in a concentration of about 1 wt. % to about 100 wt. %. In various embodiments, the coating may include about 1 wt. %, 2 wt. %, 5 wt. %, 7.5 wt. %, 8.3 wt. %, 10 wt. %, 15 wt. %, 16.7 wt. %, 20 wt. %, 25 wt. %, or about 30 wt. % silicon nitride powder or aluminum nitride powder. In some examples, the coating may include about 10 wt. % to about 20 wt. % silicon nitride or aluminum nitride. In at least one example, the coating includes about 15 wt. % silicon nitride or aluminum nitride. In some embodiments, silicon nitride or aluminum nitride may be embedded in (as a filler) or on the surface of the article in a concentration of about 1 wt. % to about 100 wt. %. In various embodiments, the object may include about 1 wt. %, 2 wt. %, 5 wt. %, 7.5 wt. %, 8.3 wt. %, 10 wt. %, 15 wt. %, 16.7 wt. %, 20 wt. %, 25 wt. %, 30 wt. %, 33.3 wt. %, 35 wt. %, 40 wt. %, 50 wt. %, 60 wt. %, 70 wt. %, 80 wt. %, 90 wt. %, to 100 wt. % silicon nitride or aluminum nitride. In some examples, the silicon nitride or aluminum nitride may be on the surface of the article at a concentration of about 10 wt. % to about 20 wt. %. In at least one example, the silicon nitride or aluminum nitride may be on the surface of the article at a concentration of about 15 wt. %. In some aspects, the concentration of silicon nitride or aluminum nitride may depend on the substrate material of the object.

In some embodiments, the article is PPE. In some aspects, the article is a body cover, a head cover, a shoe cover, a face mask, a face and eye protector, or gloves. In some aspects, the article is operable to inactivate a SARS-CoV-2 virus when the article contacts the virus.

### II. Nitride Based Antipathogenic Compositions and Devices and Methods of Use Thereof

Provided herein are antipathogenic compositions and devices that include a nitride for the inactivation of viruses, bacteria, and fungi. As used herein, a nitride is a compound of nitrogen where nitrogen has a nominal oxidation state of between −3 and +5. Non-limiting examples of suitable nitrides include silicon nitride, aluminum nitride, boron nitride, chromium nitride, cerium nitride, hafnium nitride, lanthanum nitride, phosphorus nitride, sulfur nitride, tantalum nitride, titanium nitride, vanadium nitride, yttrium nitride, zirconium nitride, or combinations thereof. Nitrides can have high intrinsic nitrogen content. For example, silicon nitride (Si₃N₄) contains about 40 wt. % nitrogen, boron nitride (BN) has about 56 wt. % nitrogen, aluminum nitride (AlN) has 34 wt. % nitrogen, and titanium nitride (TiN) has about 22 wt. % nitrogen.

Nitrides in general may be antipathogenic due to release of nitrogen containing species when in contact with an aqueous medium, or biologic fluids and tissues. For example, the surface hydrolysis chemistry of silicon nitride may be shown as follows:

Si₃N₄+6H₂O→3SiO₂+4NH₃

SiO₂+2H₂O→Si(OH)₄

Similarly, as a second example, the surface hydrolysis chemistry of boron nitride may be shown as follows:

BN+3H₂O→B₂O₃+2NH₃

Furthermore, in the presence of water, ammonia and ammonium exist in a pH-dependent equilibrium as follows:

NH₃⁺H₂O [symbol] NH₄⁺(aq)+OH⁻(aq)

The elution of ammonia, its protonation to ammonium and hydroxide ions, and the attendant increase in pH, inactivates viruses, bacteria, and fungi. Similar to silicon nitride, other nitride materials may exhibit antipathogenic properties due to surface hydrolysis of nitrogen and the consequential formation of ammonia and ammonium. It was previously believed that only β-silicon nitride exhibited antipathogenic properties. However, it was surprisingly found that other nitride materials exhibit similar surface chemistry. These results were unexpected given the fact that all nitrides are essentially insoluble in water and their hydrolysis was previously considered to be insufficient to generate antipathogenic properties.

In an embodiment, the antipathogenic nitride based composition may exhibit elution kinetics that show: (i) a slow but continuous elution of ammonia from the solid state rather than from the usual gas state; (ii) no damage or negative effect to eukaryotic cells; and (iii) an intelligent elution that increases with decreasing pH.

The antipathogenic compositions and devices disclosed herein can comprise one or more of aluminum nitride, boron nitride, chromium nitride, cerium nitride, hafnium nitride, lanthanum nitride, phosphorous nitride, sulfur nitride, tantalum nitride, titanium nitride, vanadium nitride, yttrium nitride, and zirconium nitride. In some embodiments, the composition and devices can further comprise silicon nitride, such as α-silicon nitride. In other embodiments, the nitride may be α-silicon nitride. In still other embodiments, the antipathogenic compositions and devices may include nitride mixtures, such as a mixture of AlN and BN. It was surprisingly found that these compositions were capable of inactivating viruses.

The nitride based compositions can be powders, particulates, slurries, suspensions, coatings, films, and/or composites. In some embodiments, the compositions can comprise micrometer or nanometer particles of the nitride. The average particle size of the nitride may range from about 100 nm to about 5 μm, from about 300 nm to about 1.5 μm, or from about 0.6 μm to about 1.0 μm. In another embodiment, the composition can comprise a slurry or suspension of nitride particles.

In some embodiments, the antipathogenic composition may be a monolithic component consisting of the nitride. Such a component can be fully dense possessing no internal porosity, or it may be porous, having a porosity that ranges from about 1% to about 80%. The monolithic component may be used as a medical device or may be used in an apparatus in which the inactivation of a virus, bacteria, and/or fungi may be desired.

In another embodiment, the antipathogenic nitride based composition may be incorporated within a device or within a coating on the surface of the device to inactivate viruses, bacteria, and fungi. At least a portion of the surface of the device may be coated with coating comprising the nitride based composition. Non-limiting examples of suitable devices include orthopedic implants, spinal implants, pedicle screws, dental implants, in-dwelling catheters, endotracheal tubes, colonoscopy scopes, and other similar devices. The device or apparatus may be metallic, polymeric, and/or ceramic.

In some embodiments, the nitride may be incorporated within or applied as a coating to materials or apparatuses for antipathogenic properties such as polymers and fabrics, surgical gowns, gloves, tubing, clothing, air and water filters (e.g. filtration devices of anesthesia machines, ventilators, or CPAP machines), masks, tables such as hospital exam and surgical tables, desks, fixtures, handles, knobs, toys, and filters such as air conditioner filters, or toothbrushes.

The nitride based coating may be applied to the surface of the device as a powder. In some examples, the nitride powder may be imbedded or impregnated in at least a portion of the device. In some embodiments, the powder may be micrometric or nanometer in size. In other embodiments, the silicon nitride may be incorporated into the device. For example, a device may incorporate the nitride powder within the body of the device.

In some embodiments, the antipathogenic nitride based composition may be a slurry of nitride powder in an aqueous solution. The aqueous medium may be water, saline, buffered saline, or phosphate buffer saline. In other embodiments, the nitride base composition may be a suspension or emulsion of nitride powder and a suitable vehicle (e.g., cream, gel, or lotion) for topical application. The nitride powder may be present in the composition in a concentration of about 0.1 vol. % to about 20 vol. %. In various embodiments, the slurry may include about 0.1 vol. %, 0.5 vol. %, 1 vol. %, 1.5 vol. %, 2 vol. %, 5 vol. %, 10 vol. %, 15 vol. %, or 20 vol. % silicon nitride. In some embodiments, the concentration of the nitride may be effective to inactivate the pathogen.

In other embodiments, the nitride-based coating may be present on the surface of a device or within the device in a concentration of about 1 wt. % to about 100 wt. %. In various embodiments, the coating may include about 1 wt. %, 2 wt. %, 5 wt. %, 7.5 wt. %, 8.3 wt. %, 10 wt. %, 15 wt. %, 16.7 wt. %, 20 wt. %, 25 wt. %, 30 wt. %, 33.3 wt. %, 35 wt. %, or 40 wt. % nitride powder. In at least one example, the coating includes about 15 wt. % of the nitride. In some embodiments, nitride may be present in or on the surface of a device or apparatus in a concentration of about 1 wt. % to about 100 wt. %. In various embodiments, a device or apparatus may include about 1 wt. %, 2 wt. %, 5 wt. %, 7.5 wt. %, 8.3 wt. %, 10 wt. %, 15 wt. %, 16.7 wt. %, 20 wt. %, 25 wt. %, 30 wt. %, 33.3 wt. %, 35 wt. %, 40 wt. %, 50 wt. %, 60 wt. %, 60 wt. %, 70 wt. %, 80 wt. %, 90 wt. %, to 100 wt. % of the nitride. In some embodiments, the concentration of the nitride may be effective to inactivate the pathogen.

In some embodiments, the antipathogenic nitride based composition or device may inactivate or decrease the transmission of viruses, bacteria, and/or fungi. The viruses, bacteria, and fungi may infect mammalian cells, animal cells, and/or plant cells. Non-limiting examples of viruses that may be inactivated by the antipathogenic nitride based compositions include coronaviruses (e.g., SARS-CoV-2), rhinoviruses, influenza viruses (A, B, C, D), and Feline calicivirus. The antipathogenic nitride based compositions or devices may kill both gram-positive and gram-negative bacteria. Examples of fungi that may be lysed include, without limit, those that cause downy mildew, powdery mildew, Botrytis rot, Fusarium rot, rust, Rhizoctonia rot, Sclerotinia rot, Sclerotium rot, or other agriculturally relevant diseases.

In some embodiments, the pathogen may be on a surface of or within a human, an animal, or a plant. In other embodiments, the pathogen may be on a surface of or within an inanimate object.

Further provided herein are methods for inactivating pathogens by contacting a virus, bacteria, and/or fungus with an antipathogenic nitride based composition disclosed herein. In an embodiment, the method may include coating a device or apparatus with the nitride based composition and contacting the coated apparatus with the virus, bacterium, or fungus. In another embodiment, the method may include contacting a virus, bacteria, and/or fungus with a composition comprising a nitride based composition. The composition may be a slurry comprising nitride powder or particles. The composition may be a suspension or emulsion comprising the nitride powder.

Also provided herein is a method of treating or preventing a pathogen at a location in a human patient. For example, the pathogen may be a virus, bacterium, or fungus. The method may include contacting the patient with a device, apparatus, or composition comprising nitride based composition. The device, apparatus, or composition may include about 1 wt. % to about 100 wt. % of the nitride. In some examples, the device or apparatus may include about 1 wt. % to about 100 wt. % of the nitride on the surface of the device or apparatus. In an embodiment, the device or apparatus may be a monolithic nitride based ceramic. In another embodiment, the device or apparatus may include a nitride coating, such as a nitride powder coating. In another embodiment, the device or apparatus may incorporate the nitride into the body of the device. For example, the nitride powder may be incorporated or impregnated into the body of the device or apparatus using methods known in the art.

In some embodiments, the device or apparatus may be contacted with the patient or user for at least 1 minute, at least 5 minutes, at least 30 minutes, at least 1 hour, at least 2 hours, at least 5 hours, or at least 1 day. In at least one example, the device or apparatus may be permanently implanted in the patient. In at least one example, the device or apparatus may be worn externally by a user.

### EXEMPLARY EMBODIMENTS

Embodiment 1: A method for inactivating a pathogen comprising contacting the pathogen with a composition comprising an effective concentration of a nitride chosen from aluminum nitride, boron nitride, chromium nitride, cerium nitride, hafnium nitride, lanthanum nitride, phosphorous nitride, sulfur nitride, tantalum nitride, titanium nitride, vanadium nitride, yttrium nitride, zirconium nitride, or a combination thereof, wherein the effective concentration of the nitride inactivates the pathogen.

Embodiment 2: The method of embodiment 1, wherein the composition further comprises silicon nitride.

Embodiment 3: The method of embodiment 1, wherein the nitride is aluminum nitride and/or boron nitride.

Embodiment 4: The method of embodiment 1, wherein the composition comprises a slurry of nitride particles in an aqueous medium.

Embodiment 5: The method of embodiment 4, wherein the effective concentration of the nitride is about 0.1 vol. % to about 20 vol. %.

Embodiment 6: The method of embodiment 1, wherein the composition comprises a powder of the nitride.

Embodiment 7: The method of embodiment 6, wherein the composition is coated over at least part of a surface of a device and/or is incorporated into the device.

Embodiment 8: The method of embodiment 7, wherein the effective concentration of the nitride is about 1 wt. % to about 100 wt. %.

Embodiment 9: The method of embodiment 7 or 8, wherein the device is an orthopedic implant, a spinal implant, a pedicle screw, a dental implant, an in-dwelling catheter, an endotracheal tube, a colonoscopy scope, a surgical gown, a mask, a filter, or tubing.

Embodiment 10: The method of any one of embodiments 1 to 9, wherein the pathogen is a virus, a bacteria, or a fungus.

Embodiment 11: The method of embodiment 10, wherein the virus is a coronavirus.

Embodiment 12: The method of any one of embodiments 1 to 11, wherein the pathogen is on a surface or within a human, animal, or plant.

Embodiment 13: The method of any one of embodiments 1 to 11, wherein the pathogen is on a surface of an inanimate object.

Embodiment 14: A nitride-based composition for inactivating a pathogen, the composition comprising: a slurry of nitride particles in an aqueous medium, wherein the nitride is present at a concentration of about 0.1 vol. % to about 20 vol. %, and wherein the concentration is effective to inactivate the pathogen.

Embodiment 15: The nitride-based composition of embodiment 14, wherein the nitride is chosen from aluminum nitride, boron nitride, chromium nitride, cerium nitride, hafnium nitride, lanthanum nitride, phosphorous nitride, sulfur nitride, tantalum nitride, titanium nitride, vanadium nitride, yttrium nitride, zirconium nitride, or a combinations thereof.

Embodiment 16: The nitride-based composition of embodiment 15, wherein the composition further comprises silicon nitride.

Embodiment 17: The nitride-based composition of embodiment 15, wherein the nitride is aluminum nitride and/or boron nitride.

Embodiment 18: The nitride-based composition of embodiment 14, wherein the pathogen is a virus, a bacteria, or a fungus.

Embodiment 19: The nitride-based composition of embodiment 18, wherein the virus is a coronavirus.

Embodiment 20: The nitride-based composition of any one of embodiments 14 to 19, wherein the pathogen is on a surface or within a human, animal, or plant.

Embodiment 21: The nitride-based composition of any one of embodiments 14 to 19, wherein the pathogen is on a surface of an inanimate object.

Embodiment 22: A nitride-based device for inactivating a pathogen, the device comprising: a powder of a nitride coated over at least part of a surface of the device and/or is incorporated into the device, wherein the nitride is present at a concentration of about 10 wt. % to about 30 wt. %, and wherein the concentration is effective to inactivate the pathogen.

Embodiment 23: The nitride-based device of embodiment 22, wherein the nitride is chosen from aluminum nitride, boron nitride, chromium nitride, cerium nitride, hafnium nitride, lanthanum nitride, phosphorous nitride, sulfur nitride, tantalum nitride, titanium nitride, vanadium nitride, yttrium nitride, zirconium nitride, or a combinations thereof.

Embodiment 24: The nitride-based device of embodiment 23, wherein the device further comprises silicon nitride.

Embodiment 25: The nitride-based device of embodiment 23, wherein the nitride is aluminum nitride and/or boron nitride.

Embodiment 26: The nitride-based device of embodiment 22, wherein the pathogen is a virus, a bacteria, or a fungus.

Embodiment 27: The nitride-based device of embodiment 26, wherein the virus is a coronavirus.

Embodiment 28: The nitride-based device of any one of embodiment 22 to 27, wherein the pathogen is on a surface or within a human, animal, or plant.

Embodiment 29: The nitride-based device of any one of embodiment 22 to 27, wherein the pathogen is on a surface of an inanimate object.

Embodiment 30: A method for inactivating a pathogen comprising contacting the pathogen with a slurry comprising an effective concentration of α-silicon nitride, wherein the effective concentration of the nitride inactivates the pathogen, and wherein the effective concentration of α-silicon nitride in the slurry is about 15% w/v.

### EXAMPLES

### Example 1: Rapid Inactivation of SARS-CoV-2 by Silicon Nitride or Aluminum Nitride

The present study compared the effects of exposing the SARS-CoV-2 virus to aqueous suspensions of silicon nitride (Si₃N₄) particles, aluminum nitride (AlN) particles, and two controls, (i.e., a suspension of copper (Cu) particles (positive control) and no antiviral agent (negative control)). Cu was chosen as a positive control because of its well-known ability to inactivate a variety of microbes, including viruses.

### Preparation of Test Materials

Si₃N₄, Cu, and AlN powders were acquired from commercial sources. Si₃N₄ powder (nominal composition of 90 wt. % Si₃N₄, 6 wt. % Y₂O₃, and 4 wt. % Al₂O₃) was prepared by aqueous mixing and spray-drying of the inorganic constituents, followed by sintering of the spray-dried granules (˜1700° C. for ˜3 h), hot-isostatic pressing (1600° C., 2 h, 140 MPa in N₂), aqueous-based comminution, and freeze-drying. The resulting powder had an average particle size of 0.8±1.0 μm. As-received Cu powder (USP grade 99.5% purity) granules were comminuted to achieve a particle size comparable to the Si₃N₄. AlN powder had an average particle size of 1.2±0.6 μm as-received, which was comparable to Si₃N₄.

### Preparation of Mammalian and Viral Cells

VeroE6/TMPRSS2 mammalian cells were used in the viral assays. Cells were grown in Dulbecco's modified Eagle's minimum essential medium (DMEM) supplemented with G418 disulfate (1 mg/ml), penicillin (100 units/mL), streptomycin (100 μg/mL), 5% fetal bovine serum, and maintained at 37° C. in a 5% CO2/95% in a humidified atmosphere. The SARS-CoV-2 viral stock was propagated using VeroE6/TMPRSS2 cells at 37° C. for 2 days. Viral titers were assayed by a median tissue culture infectious dose (TCID₅₀).

### Example 2

### Virus Assays

Fifteen weight percent (15 wt. %) of the Si₃N₄, Cu, and AlN powders were separately dispersed in 1 mL of PBS(−), followed by the addition of the viral suspension (2×10⁵ TCID₅₀ in 20 μL). Due to the higher density of the Cu powder, its volumetric fraction was approximately one-third of the Si₃N₄. Mixing was performed for 1 and 10 minutes by slow manual rotation at 4° C. After exposure, the powders were pelleted by centrifugation (2400 rpm 2 minutes) followed by filtration through a 0.1 μm media. Supernatants were collected and subjected to TCID₅₀ assays, real-time RT-PCR testing, and fluorescence imaging. Experiments were performed in triplicate including sham supernatants without the antiviral powders. A confluent monolayer of VeroE6/TMPRSS2 cells in a 96-well plate was inoculated with 50 μL/well of each virus suspension in a tenfold serial dilution with 0.5% FBS DMEM (i.e., maintenance medium). Viral adsorption at 37° C. for 1 hour was made with tilting every 10 minutes. Afterwards, 50 μL/well of the maintenance medium was added. The plate was incubated at 37° C. in a 5% CO2/95% humidified atmosphere for 4 days. The cytopathic effect (CPE) of the infected cells was observed under a phase-contrast microscope. The cells were subsequently fixed by adding 10 μL/well of glutaraldehyde followed by staining with 0.5% crystal violet. The TCID₅₀ was calculated according to the Reed-Muench method.

### Viral RNA Assay

After exposure to the powders, 140 μL of the supernatants were used for viral RNA extraction. RNA was also extracted from the surfaces of the centrifuged and filtered powders. RNA purification was performed by using a QIAamp Viral RNA Mini kit. An aliquot of 16 μL of isolated RNA was reverse-transcribed using ReverTra Ace® qPCR RT Master Mix. Quantitative real-time PCR was performed using a Step-One Plus Real-Time PCR system primers/probes for two specific viral N gene sets. Each 20 μL reaction mixture contained 4 μL of cDNA, 8.8 μmol of each primer, 2.4 μmol of the probe, and 10 μL GoTaq Probe qPCR master mix. The amplification protocol consisted of 50 cycles of denaturation at 95° C. for 3 seconds and annealing and extension at 60° C. for 20 seconds.

### Immunochemical Fluorescence Assay

Vero E6/TMPRSS2 cells on cover glass were inoculated with 200 μL of virus supernatant. After viral adsorption at 37° C. for 1 hour, the cells were incubated with the maintenance medium in a CO2 incubator for 7 hour. For the detection of infected cells, these cells were washed with TBS (20 mM Tris-HCl pH 7.5, 150 mM NaCl) and fixed with 4% PFA for 10 min at room temperature (RT) followed by membrane permeabilization with 0.1% Triton X in TBS for 5 minutes at RT. The cells were blocked with 2% skim milk in TBS for 60 minutes at RT and stained with anti-SARS Coronavirus envelope (Rabbit) antibody (Dilution=1:100) for 60 minutes at RT. After washing with a buffer, cells were incubated with an Alexa 594 Goat Anti-Rabbit IgG (H+L) (1:500) and Alexa 488 Phalloidin (1:50) for 60 min at RT in the dark. ProLong™ Diamond Antifade Mountant with DAPI was used as a mounting medium. The staining was observed under a fluorescent microscope BZX710. The total cell and infected cell counts were obtained using the Keyence BZ-X Analyzer.

### Raman Spectroscopy Assay

Vero E6/TMPRSS2 cells were infected with 200 μL of each virus suspension onto glass sites. After viral adsorption at 37° C. for 1 hours, the infected cells were incubated with the maintenance medium in a CO₂ incubator for 4 hours and fixed with 4% paraformaldehyde for 10 minutes at RT. After washing with distilled water twice, infected cells were air-dried and in situ analyzed using a Raman microprobe spectrometer. Raman spectra were collected using a highly sensitive spectroscope with a 20× optical lens. It operated in microscopic measurement mode with confocal imaging in two dimensions. A holographic notch filter within the optical circuit was used to efficiently achieve a spectral resolution of 1.5 cm-1 via a 532 nm excitation source operating at 10 mW. Raman emissions were monitored using a single monochromator connected to an air-cooled charge-coupled device (CCD) detector 1024×256 pixels). The acquisition time was fixed at 10 seconds. Thirty spectra were collected and averaged for each analysis time-point. Raman spectra were deconvoluted into Gaussian-Lorentzian sub-bands using commercially available software.

### Statistical Analysis

The Student's t-test determined statistical significance for n=3 and at a p-value of 0.01 using Prism software.

### Example 3

### Median Tissue Culture Infection Dose

TCID₅₀ assay results for the 15 wt. % Si₃N₄, Cu, and AlN powders are shown in FIGS. 1A-1D. Inactivation times of 1 and 10 minutes are shown in FIGS. 1A and 1B as well as FIGS. 1C and 1D, respectively. Relative to the negative control, all three powders were effective in inactivating SARS-CoV-2 virions (>99%) for the two exposure times.

### RNA Gene Fragmentation

To examine whether viral RNA was fragmented from exposure to both the supernatants and powders, RT-PCR tests were conducted on the N gene sets of the virus' RNA. The results are shown in FIGS. 2A and 2B as well as FIGS. 2C and 2D for 1- and 10-minute exposures, respectively. Again, in comparison to the negative control at 1 minute of exposure to the supernatants, almost complete fragmentation of the RNA was observed for Cu while significant damage was caused by AlN and to a lesser extent by Si₃N₄. After 10-minute exposure to the supernatants, substantial cleavage of the RNA was seen for all three materials. While Cu still showed the most fragmentation, Si₃N₄ demonstrated similar effectiveness, and AlN was essentially identical to the 1-minute exposure condition. Viral RNA was virtually undetectable for all three materials based on extracted RNA from the pelleted powders at 1-minute of exposure (cf., FIGS. 2A and 2B). This result suggests that the decrease of viral RNA in the supernatant was not because of adherence of the RNA to the powders, but rather due to direct degradation.

### Immunofluorescence Testing

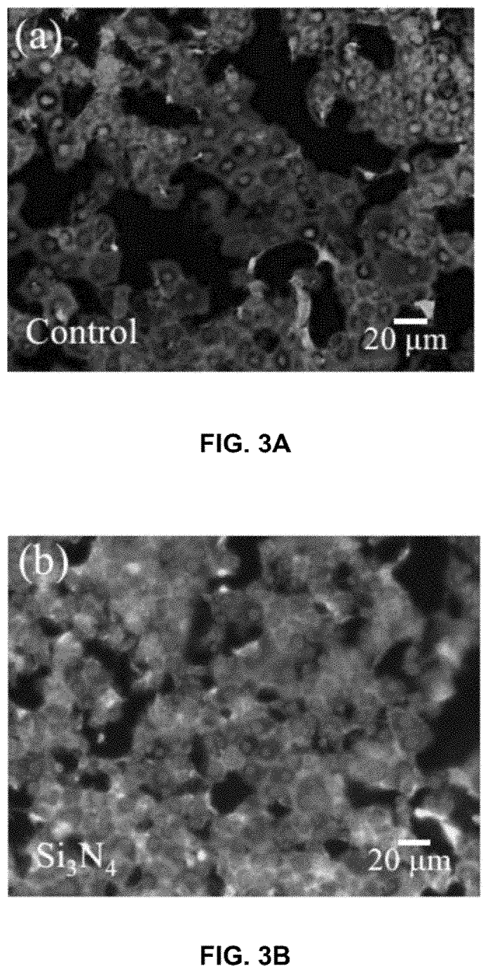

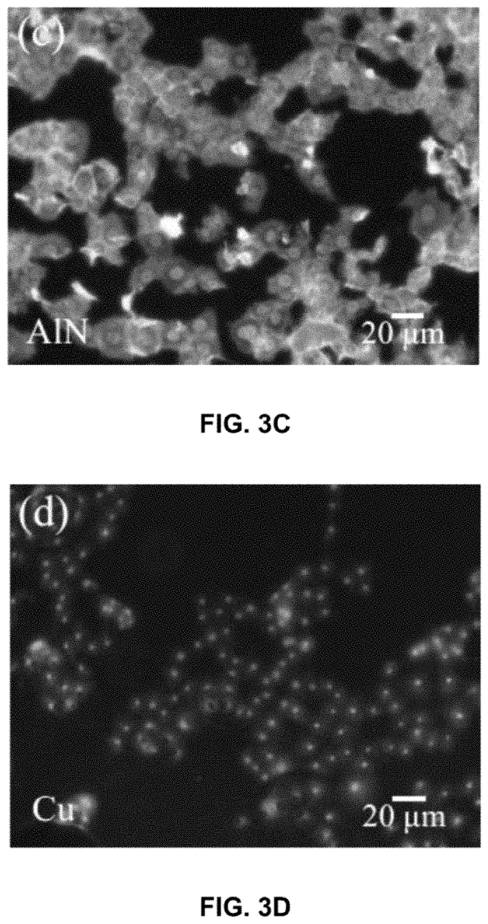

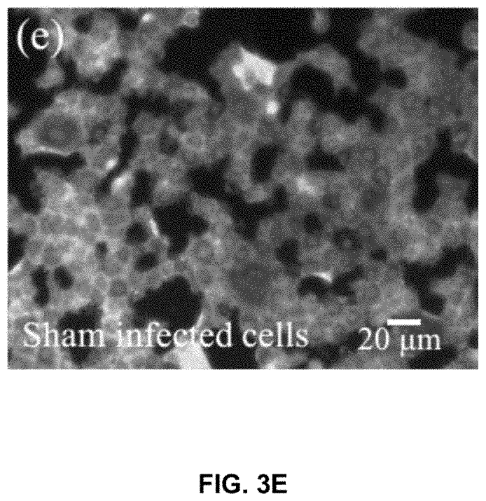

Immunofluorescence imaging using anti-SARS coronavirus envelope antibody (red), Phalloidin that stains F-actin in viable cells (green), and DAPI for cell nuclear staining (blue) was then used to confirm the TCID₅₀ assay and gene fragmentation results. FIGS. 3A-3D show fluorescence micrographs representative of the VeroE6/TMPRSS2 cell populations that were inoculated with supernatants of (a) unexposed virions (i.e., negative control) and 10-minute-exposed virions of (b) Si₃N₄, (c) AlN, and (d) Cu. FIG. 3E shows cells that were not inoculated with the virus (labeled as “sham-infected” cells. The red-colored spots in the negative control (FIG. 3A) demonstrated that the virions had entered and hijacked the Vero6E cells' metabolism. This contrasts with the sham-infected cells (FIG. 3E) which showed normal metabolic function.

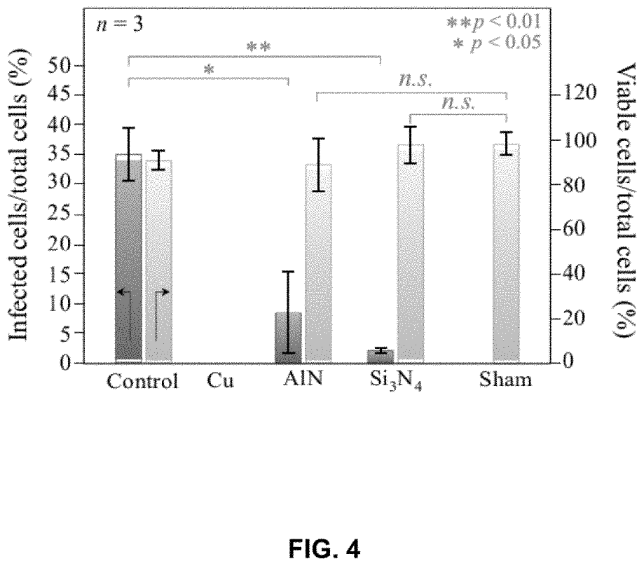

Remarkably, cells inoculated with supernatants from Si₃N₄ and, to a lesser extent, from AlN demonstrated near-normal function with few infections. Conversely, cells inoculated with the Cu supernatant were essentially dead (i.e., a complete lack of F-actin, FIG. 3D), although based on the bluish-red stains present in the nuclei, they may have been viable premortem because virions appear to have hijacked some nuclei. This suggests that cellular lysis was not only the result of the viral infection but also due to toxic effects from intracellular free Cu ions. Quantification of the colorimetric results from FIGS. 3A-3E is provided in FIG. 4. These data demonstrate that about 35% of the viable VeroE6 cells from the negative control were infected by virions, whereas only 2% and 8% of cells inoculated with supernatants from Si₃N₄ and AlN were infected, respectively. Quantitative evaluation of the cells inoculated with the Cu supernatant could not be assessed due to their premature death.

### Raman Spectroscopy

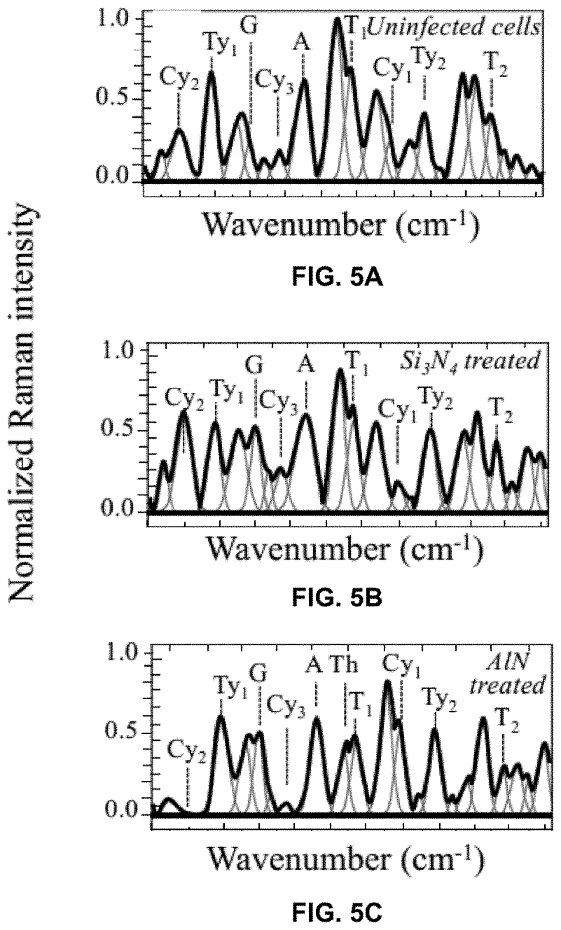

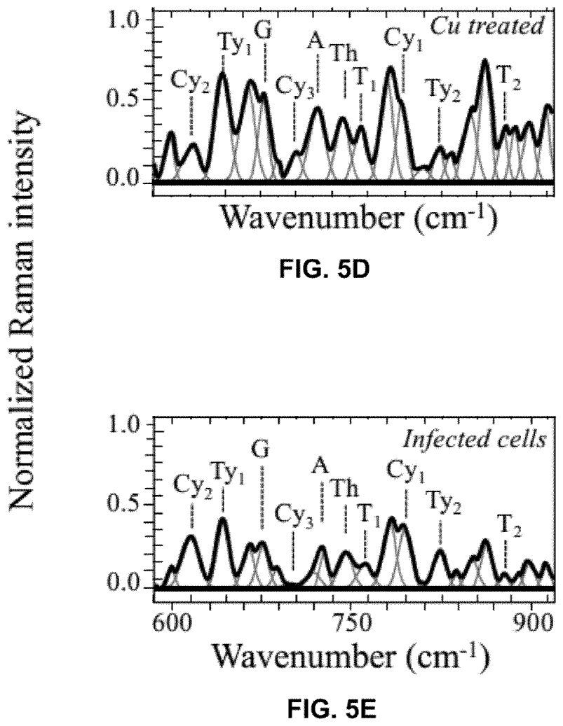

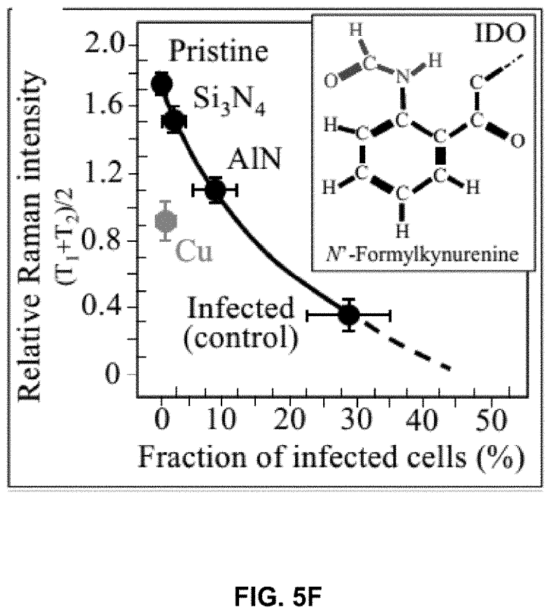

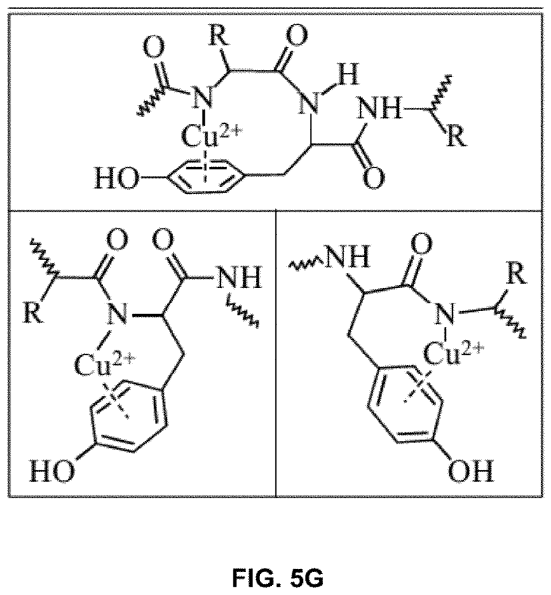

Raman spectroscopy examined VeroE6 cells exposed to the various supernatants to assess biochemical cellular changes due to infection and ionic (i.e., Cu and Al) toxicity. FIGS. 5A-5G show Raman spectra in the frequency range 700-900 cm-1 for (a) uninfected VeroE6/TMPRSS2 cells, and cells inoculated with supernatants containing virions exposed for 10-minutes to (b) Si₃N₄, (c) AlN, (d) Cu (positive control), and (e) no antiviral compounds (negative control). Of fundamental importance are the vibrational bands of ring breathing and H-scissoring of the indole ring of tryptophan (at 756 and 875 cm-1, labeled as T1 and T2, respectively). Tryptophan plays a vital role in protein synthesis and the generation of molecules for various immunological functions. Its stereoisomers serve to anchor proteins within the cell membrane and its catabolites possess immunosuppressive functions. The catabolism of tryptophan is triggered by a viral infection. This occurs via the enzymatic activity of indoleamine-2,3-dioxygenase (IDO) which protects the host cells from an over-reactive immune response. IDO reduces tryptophan to kynurenine and then to N′-formyl-kynurenine. An increase in IDO activity depletes tryptophan. Consequently, the intensity of the tryptophan bands (T1 and T2) is an indicator of these biochemical changes. Except for the Cu-treated sample, the data presented in FIG. 5F show an exponential decline in the combined tryptophan bands that correlates with the fraction of infected cells. (The chemical structure of N′-formyl-kynurenine is given in the inset for clarity.) The anomaly for copper provides further evidence of its toxicity. The VeroE6 cells consumed tryptophan to reduce Cu²⁺ and stabilize it as Cu⁺.

The Raman signals due to ring-stretching vibrations of adenine, cytosine, guanine, and thymine were found at 725, 795, 680, and 748 cm-1, and are labeled as A, Cy1, G, and Th, respectively, in FIGS. 5A-5E). These bands were preserved after virus exposure. However, there was an anomaly for lines representative of tyrosine at 642 and 832 cm-1 labeled as Ty1 and Ty2, respectively for cells infected with Cu-exposed virions. The ring-breathing band Ty2 of tyrosine was very weak compared to the other samples (cf. FIG. 5D with FIG. 5B). Conversely, the C—C bond-related Ty1 signal remained strong. This suggests that the aromatic ring of tyrosine chelated the Cu ions. This explains why only the tyrosine ring-breathing mode was reduced while the C—C signal remained unaltered. Three possible Cu(II) chelating conformations in tyrosine are given in FIG. 5G.

For VeroE6 cells exposed to virions treated with AlN (FIG. 5C), the tryptophan T1 and T2 bands were preserved, but the bands at 615 and ˜700 cm-1 due to ring bending in DNA cytosine (labeled as Cy2 and Cy3, respectively, in FIGS. 5A-5E) almost vanished. Their disappearance is due to either progressive internucleosomal DNA cleavage or from the formation of complexes, and both are related to toxicity. The loss of the cytosine signals is interpreted as a toxic effect by Al ions, although it is far less critical than copper. Al₃⁺ interacts with carbonyl O and/or N ring donors in nucleotide bases and selectively binds to the backbone of the PO2 group and/or to the guanine N-7 site of the G-C base pairs by chelation.

Unlike exposure of the VeroE6 cells to Cu and AlN supernatants, which resulted in moderate to severe toxicity, Si₃N₄ invoked no modifications of tryptophan, tyrosine, and cytosine. The morphology of the spectrum for the Si₃N₄ viral supernatant closely matched that of the uninfected sham suspension (cf. FIGS. 5A and 5B).

### Example 4: Biofilm Assay of Aluminum Nitride and Boron Nitride Materials

This study was conducted to compare Staphylococcus epidermidis biofilm growth on biomaterial variations of silicon nitride and other ceramic materials, along with PEEK as a positive control. The ceramic materials under observation included as-fired silicon nitride (AFSN), aluminum nitride (AlN), two grades of boron nitride (BN: AX05BN and PCBN1000), and Shapal (a mixture of AlN and BN).

Replicates of three were chosen for each sample material at time points of 24 and 48 h for basic statistical analysis. Samples were prepared by cleaning them in alcohol under ultrasonic agitation for 5 minutes, followed by a DI water rinse using ultrasonic agitation for 5 minutes, then sterilizing the samples by UV-C light exposure for 30 minutes on each side, and allowing samples to rest for at least 60 minutes before inoculation.

A bacterial medium was prepared by combining 7% glucose, 1×PBS, and 10% human plasma, then inoculating it with a small aliquot of Staphylococcus epidermidis. An initial absorbance value was taken using a spectrophotometer. The medium was then placed in a shaking incubator at 37° C. and 175 rpm for six hours where the bacteria was allowed to proliferate until an absorbance value of 0.05 AU was achieved (corresponding to 10⁵ cells/mL per a previously generated growth curve).

Each sterile sample was placed in a well plate and 7 mL of liquid culture was added. Well plates were placed in a shaking incubator at 37° C. and 125 rpm for 24 hours. At t=24 hours, the liquid culture was removed and 7 mL of fresh media was added. The well plates were placed back in shaking incubator for an additional 24 hours.

Samples were removed at appropriate time points (24 or 48 hours) and rinsed in 5 mL of 1×PBS in a fresh well plate in the shaking incubator for 2 minutes at 125 rpm. Each sample was dip-rinsed in fresh 1×PBS. Samples were placed in 10 mL 1×PBS in a 50 mL centrifuge tube and vortexed vigorously for 2 minutes.

Surface area measurements were obtained using disc diameters, widths, and weights. The number of colonies on each Petrifilm were counted, recorded, and compared.

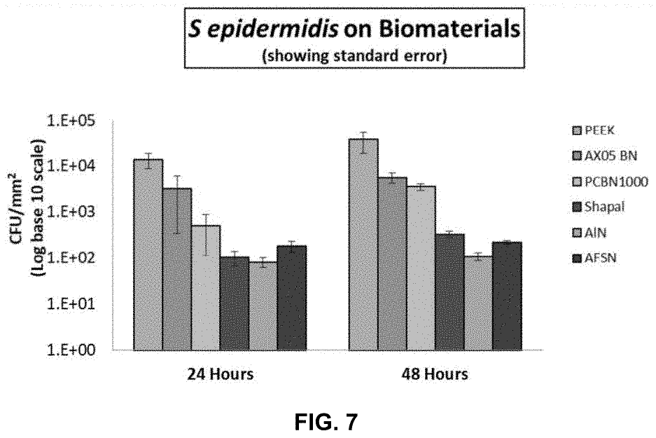

Each sample's biofilm solution was serially diluted in 1×PBS, resulting in concentrations 1×, 1/10×, 1/100×, 1/1,000×, and 1/10,000× of the original sample. Each dilution was plated onto a Petrifilm to allow the lowest countable dilution to be used in data comparison for each sample. After growing in the incubated environment of 37° C. for 24 hours, the bacteria formed colonies, referenced as colony forming units (CFU). Once obtaining the CFU count, the CFUs per sample were calculated by multiplying the count by the appropriate dilution factor. The total CFUs were then divided by surface area to accurately compare samples of differing sizes, so the final data reported is in units of CFU/mm². Statistical analyses were performed and graphed. Table 1 and FIG. 7 show the mean for each material at both 24 and 48 hours. A statistical analysis was performed by applying a heteroscedastic two-tailed t-test with a 95% confidence interval to all samples.

TABLE 1

| | Mean CFU/mm² (standard error) | | | | | |
|---|---|---|---|---|---|---|
| | PEEK | AFSN | AlN | Shapal | AX05 BN | PCBN1000 |
| 24 hours | 13645 (5048) | 182 (46) | 81 (19) | 102 (36) | 3248 (2901) | 501 (388) |
| 48 hours | 36818 (18059) | 220 (21) | 108 (22) | 329 (57) | 5665 (1455) | 3561 (574) |

At 24 hours, PEEK's bacterial growth was more than an order of magnitude greater than As-Fired β-Si₃N₄ (AFSN), AlN, and Shapal, which were all p<0.05. There was a statistical difference found between PEEK and all materials except for AX05BN. AX05BN was also found to have a statistically significant difference in biofilm growth as compared to all other materials. Biofilm growth comparisons between AlN, Shapal, AFSN, and PCBN1000 were not significantly different overall. It can be concluded that for the 24-hour time point, AlN, Shapal, PCBN1000, and AFSN contain the most efficient antibacterial properties.

At 48 hours, the serial dilution and plating procedure described in was performed on the remaining samples. The biofilm growth on PEEK was consistent with the results found at 24 hours, as the CFU count remained at least an order of magnitude greater than any of the ceramic materials. The representative p-value for this comparison did not reflect the visual difference due to one PEEK sample outlier. Because of expected biological variability, this outlier does not change the conclusions made from the overall results. A significant difference was found for both AX05BN and PCBN1000 in comparison to AFSN, Shapal, and AlN. The p-values for AFSN, AlN, and Shapal, when compared to AX05BN, were p=0.032, p=0.031, and p=0.033, respectively. The 48-hour results match the trends from the 24-hour. All materials had a consistent increase in biofilm growth over time.

At both the 24- and 48-hour time points, Staphylococcus epidermidis was found to form a significantly denser biofilm on PEEK than any of the ceramic variants, as expected. AX05BN did not perform as efficiently as the other ceramic materials evaluated in this experiment, as the resulting CFU counts were not significantly different than PEEK. Shapal, AlN, and AFSN had similar biofilm growth, highlighting their ability to resist S. epidermidis surface adherence and colonization. Although their bacterial resistance was quite similar, AlN outperformed PEEK, AX05BN, PCBN1000, Shapal and AFSN in resisting biofilm formation at both the 24- and 48-hour time point evaluations. Shapal and AFSN closely followed AlN in their antibacterial properties.

These results support previous conclusions that ceramic materials significantly outperform PEEK in biofilm resistance. Comparison within the ceramic samples shows that AX05BN is significantly less efficient at resisting bacterial adherence than the other ceramic compositions. Although there was not a significant difference seen between Shapal, AlN, and AFSN, AlN had slightly less biofilm formation at both time points.

### Example 5: Rapid Inactivation of SARS-CoV-2 by Alpha-Silicon Nitride and Beta-Silicon Nitride

Two silicon nitride powders were prepared. The β-silicon nitride powder had a nominal composition of 90 wt. % β-Si₃N₄, 6 wt. % yttira (Y₂O₃), and 4 wt. % alumina (Al₂O₃). The powders were each prepared by aqueous mixing and spray-drying of the inorganic constituents, followed by sintering the spray-dried granules at about 1700° C. for about 3 hours. Next, the sintered granules were hot-isostatic pressed at about 1600° C. for 2 hours at 140 MPa in nitrogen. The pressing was followed by aqueous based comminution and freeze-drying.

The α-silicon nitride powder was 98 wt. % pure Si₃N₄ with about 2 wt. % SiO₂. It was prepared by heating commercially available high-purity α-silicon nitride at about 300° C. for about 1 hour in air and then cooling it to room temperature.

To perform the antiviral testing, the Washington State variant of the SARS-CoV-2 virus was diluted in DMEM growth media to a concentration of 2×10⁴ virions/m L. Four mL of the diluted virus solution were then added to tubes containing either α- or β-silicon nitride powders at 15 wt. %/vol (w/v). The virus without Si₃N₄ was processed in parallel as a control. The tubes were vortexed for 30 seconds to ensure adequate contact and then placed on a tube revolver for 30 minutes. The samples were centrifuged, and the supernatant collected and filtered through a 0.2 μm filter. The RNA in the remaining infectious virus within the clarified supernatant was isolated along with the pellets and quantified by RT-qPCR methods.

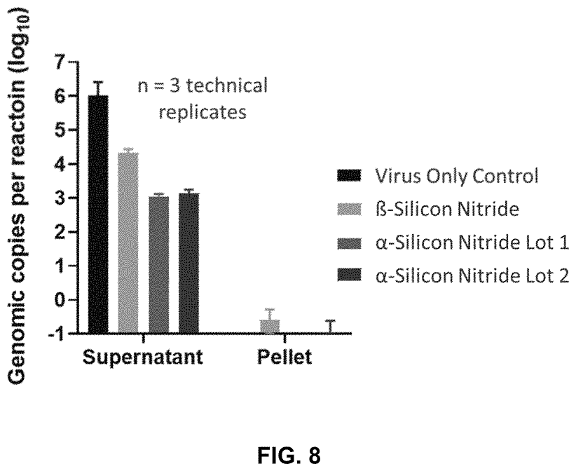

The supernatants were also subjected to plaque assay test methods. The results, provided in FIG. 8, showed that the genomic copies of virus only control had a concentration of about 1×10⁶/mL, whereas one of the β-silicon nitride powders demonstrated a reduction in virions to approximately 4.3×10⁴/mL (95.9%) and two different lots of α-silicon nitride showed viral reductions to approximately 1×10³/mL (99.9%). In this same test series, the pelleted samples from the two α-silicon nitrides were found to be completely free of live virions.

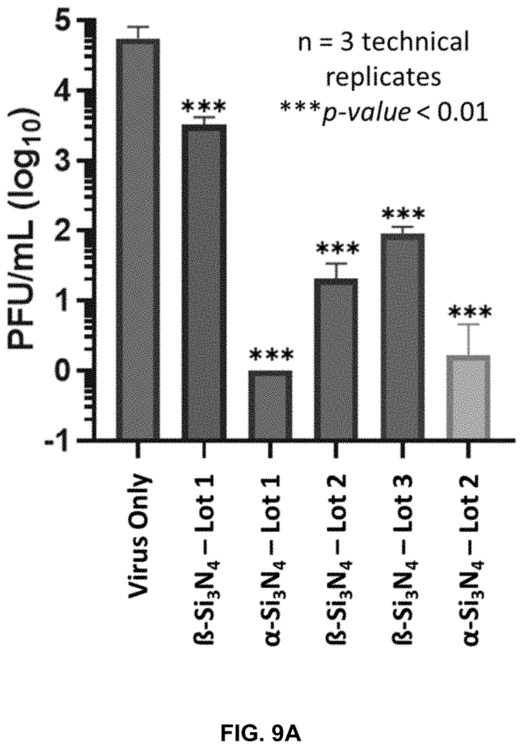

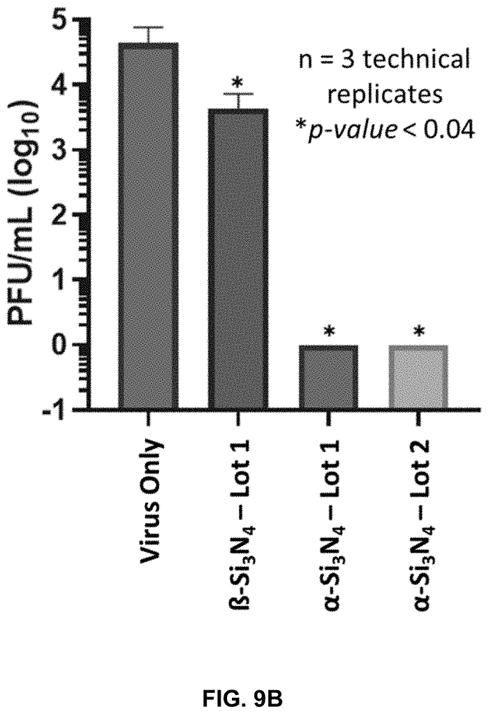

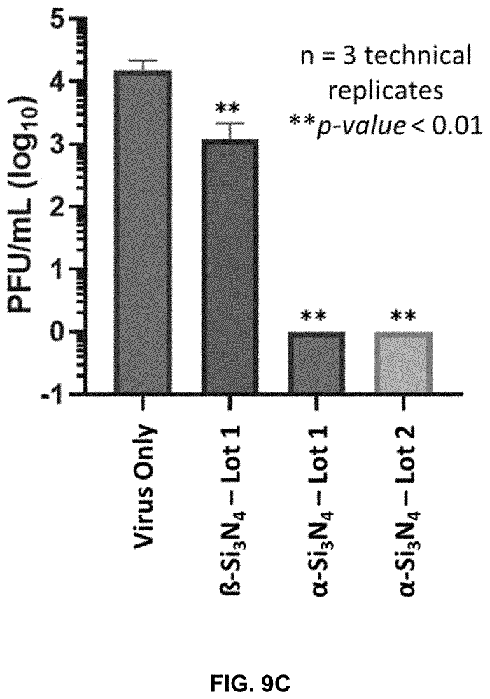

The plaque assay test results are provided in FIG. 9A. These data showed a reduction in viral activity for the β-silicon nitride powders that ranged from 1.25 to 3.5 log₁₀ (˜93% to 99.97%) whereas the α-silicon nitride powders demonstrated >4.5 log₁₀ reduction (>99.997%). It is believed that variations in the surface hydrolysis of the β-silicon nitride powders led to the observed range of results. However, comparing the RT-qPCR and plaque assay methods suggests that the SARS-CoV-2 virions are not being pelleted with the silicon nitride and that their RNA structure is being damaged when incubated with silicon nitride.

Using the same powders and methods as described above, plaque assay tests were conducted on the effectivity of α- and β-silicon nitride powders against the South African variant of the SARS-CoV-2 virus. Results are shown in FIG. 9B. The α-silicon nitride proved to be very effective with >4.5 log₁₀ reduction (99.9997%) in the virus and the β-silicon nitride powder showing approximately a 1-log₁₀ reduction (˜90%).

Similarly, using the same materials and procedures as given above, plaque assays were conducted on the effectivity of α- and β-silicon nitride powders against the British variant of the SARS-CoV-2 virus. Results are provided in FIG. 9C. The α-silicon nitride powder reduced viral counts by about 4-log₁₀ (99.99%) and the β-silicon nitride by about 1-log₁₀ (˜90%).

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for inactivating a pathogen comprising contacting the pathogen with a composition comprising an effective concentration of a nitride chosen from aluminum nitride, boron nitride, chromium nitride, cerium nitride, hafnium nitride, lanthanum nitride, phosphorous nitride, sulfur nitride, tantalum nitride, titanium nitride, vanadium nitride, yttrium nitride, zirconium nitride, or a combination thereof, wherein the effective concentration of the nitride inactivates the pathogen.

2. The method of claim 1, wherein the composition further comprises silicon nitride.

3. The method of claim 1, wherein the nitride is aluminum nitride and/or boron nitride.

4. The method of claim 1, wherein the composition comprises a slurry of nitride particles in an aqueous medium.

5. The method of claim 4, wherein the effective concentration of the nitride is about 0.1 vol. % to about 20 vol. %.

6. The method of claim 1, wherein the composition comprises a powder of the nitride.

7. The method of claim 6, wherein the composition is coated over at least part of a surface of a device and/or is incorporated into the device.

8. The method of claim 7, wherein the effective concentration of the nitride is about 1 wt. % to about 100 wt. %.

9. The method of claim 7, wherein the device is an orthopedic implant, a spinal implant, a pedicle screw, a dental implant, an in-dwelling catheter, an endotracheal tube, a colonoscopy scope, a surgical gown, a mask, a filter, or tubing.

10. The method of any one of claim 1, wherein the pathogen is a virus, a bacteria, or a fungus.

11. The method of claim 10, wherein the virus is a coronavirus.

12. The method of any one of claim 1, wherein the pathogen is on a surface or within a human, animal, or plant.

13. The method of any one of claim 1, wherein the pathogen is on a surface of an inanimate object.

14. A method for inactivating a pathogen comprising contacting the pathogen with a slurry comprising an effective concentration of α-silicon nitride, wherein the effective concentration of the nitride inactivates the pathogen, and wherein the effective concentration of α-silicon nitride in the slurry is about 15% w/v.
